(19) United States
(12) Patent Application Publication
(10) Pub. No.: US 2026/0140048 A1
(43) Pub. Date: May 21, 2026

(54) REGIONAL SOIL MOISTURE MONITORING METHOD BASED ON AIRBORNE SPECTRUM RECONSTRUCTED OPTICAL SATELLITE REMOTE SENSING DATA

(71) Applicant: Northwest A&F University, Xianyang (CN)

(72) Inventors: Shikun Sun, Xianyang (CN); Yifei Yao, Xianyang (CN); Zugui Huang, Xianyang (CN); Furong Yang, Xianyang (CN)

(21) Appl. No.: 19/315,677

(22) Filed: Sep. 1, 2025

Related U.S. Application Data
(63) Continuation of application No. PCT/CN2025/111851, filed on Jul. 31, 2025.

(30) Foreign Application Priority Data
Nov. 20, 2024 (CN) 202411663640.0

(51) Int. Cl. G01N 21/31; G01N 21/17; G01N 33/24; G06T 7/00

(52) U.S. Cl. CPC G01N 21/31; G01N 33/246; G06T 7/0002; G01N 2021/1793; G01N 2201/0214; G06T 2207/10032; G06T 2207/20081; G06T 2207/30188

(57) ABSTRACT

Provided is a regional soil moisture monitoring method based on airborne spectrum reconstructed optical satellite remote sensing, which includes the following steps: acquiring satellite remote sensing data of a to-be-monitored area, and acquiring unmanned aerial vehicle (UAV) remote sensing data of a local area in the to-be-monitored area; performing, according to the UAV remote sensing data of the local area, spectral reconstruction on the satellite remote sensing data of the to-be-monitored area, to thereby obtain reconstructed satellite spectral data of the satellite remote sensing data; and inputting the reconstructed satellite spectral data into a pre-trained soil moisture inversion model to obtain a soil moisture content of the to-be-monitored area. The method can accurately and quickly monitor soil moisture.

### TECHNICAL FIELD

The present disclosure relates to the field of soil moisture monitoring technologies, and particularly to a regional soil moisture monitoring method based on airborne spectrum reconstructed optical satellite remote sensing.

### BACKGROUND

Soil moisture is usually quantified by soil moisture content, which has important research value in crop growth monitoring, regional drought monitoring and early warning, efficient use of water resources, precision irrigation and smart agriculture.

At present, the use of optical satellite remote sensing can achieve monitoring of soil moisture in a large-scale irrigation area. However, due to limitations in spatial resolution and the dynamic changes in vegetation coverage (surface spatial heterogeneity), the accuracy of soil moisture monitoring remains relatively low. In contrast, low-altitude unmanned aerial vehicle (UAV) remote sensing has the characteristics of ultra-high altitude resolution and faster response, and can capture rich surface information, which is more accurate than the optical satellite remote sensing in monitoring soil moisture. However, for a large-scale irrigation area, it is time-consuming and laborious to collect remote sensing data of UAV covering the whole large-scale irrigation area, and the timeliness of soil moisture monitoring products cannot be guaranteed.

Therefore, there is an urgent need for a method that can accurately and quickly monitor soil moisture.

### SUMMARY

Based on this, it is necessary to provide a regional soil moisture monitoring method based on airborne spectrum reconstructed optical satellite remote sensing, which can accurately and quickly monitor soil moisture.

The present disclosure provides technical solutions as follows.

In a first aspect, an embodiment of the present disclosure provides a regional soil moisture monitoring method based on airborne spectrum reconstructed optical satellite remote sensing, which includes the following steps:
acquiring satellite remote sensing data of a to-be-monitored area, and acquiring unmanned aerial vehicle (UAV) remote sensing data of a local area in the to-be-monitored area;
performing, according to the UAV remote sensing data of the local area, spectral reconstruction on the satellite remote sensing data of the to-be-monitored area, to obtain reconstructed satellite spectral data of the satellite remote sensing data; and
inputting the reconstructed satellite spectral data into a pre-trained soil moisture inversion model to obtain a soil moisture content of the to-be-monitored area.

In an embodiment, the performing, according to the UAV remote sensing data of the local area, spectral reconstruction on the satellite remote sensing data of the to-be-monitored area, to thereby obtain reconstructed satellite spectral data of the satellite remote sensing data includes:
performing up-scale conversion on the UAV remote sensing data of the local area to determine UAV spectral data of the to-be-monitored area;
determining satellite spectral data of the to-be-monitored area according to the satellite remote sensing data of the to-be-monitored area; and
reconstructing the satellite spectral data of the to-be-monitored area based on the UAV spectral data of the to-be-monitored area to obtain the reconstructed satellite spectral data of the satellite remote sensing data.

In an embodiment, spectral data includes a band reflectance and a spectral index; and the reconstructing the satellite spectral data of the to-be-monitored area based on the UAV spectral data of the to-be-monitored area to obtain the reconstructed satellite spectral data of the satellite remote sensing data includes:
reconstructing, based on the UAV spectral data of the to-be-monitored area, satellite band reflectance of the satellite spectral data of the to-be-monitored area to obtain a reconstructed satellite band reflectance of the reconstructed satellite spectral data of the satellite remote sensing data; and
determining, based on the reconstructed satellite band reflectance of the reconstructed satellite spectral data of the satellite remote sensing data and a preset transformation function, a reconstructed satellite spectral index of the reconstructed satellite spectral data of the satellite remote sensing data.

In an embodiment, a formula for calculating the reconstructed satellite band reflectance of the reconstructed satellite spectral data of the satellite remote sensing data is expressed as follows:

$${B}_{r} = {B}_{o} \{ 1 + k \cdot [ FVC / ( {B}_{u} \cdot {B}_{o} ) ] \}$$

where Bᵣ represents the reconstructed satellite band reflectance of the reconstructed satellite spectral data of the satellite remote sensing data; Bₒ represents a satellite band reflectance of the satellite spectral data of the to-be-monitored area; FVC represents a fractional vegetation coverage corresponding to a spatial resolution scale of an optical satellite; k represents a regulation factor, k∈R; and Bᵤ represents an UAV band reflectance and an UAV spectral index.

In an embodiment, a construction process of the pre-trained soil moisture inversion model includes the following steps:
acquiring original satellite training sample data and UAV training sample data, and soil moisture contents corresponding to the original satellite training sample data and the UAV training sample data;
determining original satellite sample spectral data according to the original satellite training sample data;
performing, according to the UAV training sample data, spectral reconstruction on the original satellite sample spectral data, to obtain reconstructed satellite sample spectral data; and
training an extreme learning machine model by using the reconstructed satellite sample spectral data, to obtain the pre-trained soil moisture inversion model.

In an embodiment, the regional soil moisture monitoring method based on airborne spectrum reconstructed optical satellite remote sensing further includes:
before training the extreme learning machine model by using the reconstructed satellite sample spectral data, to obtain the pre-trained soil moisture inversion model, performing a correlation analysis between the original satellite sample spectral data and the soil moisture contents, and between the reconstructed satellite sample spectral data and the soil moisture contents, respectively.

In an embodiment, the regional soil moisture monitoring method based on airborne spectrum reconstructed optical satellite remote sensing further includes:
training the extreme learning machine model by using the original satellite sample spectral data, to obtain a soil moisture inversion model under the original satellite sample spectral data;
training the extreme learning machine model by using the reconstructed satellite sample spectral data, to obtain a soil moisture inversion model under the reconstructed satellite sample spectral data; and
performing performance testing on the soil moisture inversion model under the original satellite sample spectral data and the soil moisture inversion model under the reconstructed satellite sample spectral data, to obtain an original performance test result of the soil moisture inversion model under the original satellite sample spectral data and a performance test result of the soil moisture inversion model under the reconstructed satellite sample spectral data.

In a second aspect, an embodiment of the present disclosure provides a regional soil moisture monitoring device based on airborne spectrum reconstructed optical satellite remote sensing, which includes:
an acquisition module, configured to: acquire satellite remote sensing data of a to-be-monitored area, and acquire unmanned aerial vehicle (UAV) remote sensing data of a local area in the to-be-monitored area;
a reconstruction module, configured to: perform, according to the UAV remote sensing data of the local area, spectral reconstruction on the satellite remote sensing data of the to-be-monitored area, to obtain reconstructed satellite spectral data of the satellite remote sensing data; and
a monitoring module, configured to: input the reconstructed satellite spectral data into a pre-trained soil moisture inversion model to obtain a soil moisture content of the to-be-monitored area.

In a third aspect, an embodiment of the present disclosure provides a non-transitory computer-readable storage medium, a computer program is stored in the non-transitory computer-readable storage medium, and when the computer program is executed by a processor, the regional soil moisture monitoring method based on airborne spectrum reconstructed optical satellite remote sensing described above is implemented.

In a fourth aspect, an embodiment of the present disclosure provides a computer device, which includes a memory, a processor and a computer program stored in the memory and capable of being executed by the processor, and when the computer program is executed by the processor, the regional soil moisture monitoring method based on airborne spectrum reconstructed optical satellite remote sensing described above is implemented.

At least one of the technical solutions provided in the present disclosure can achieve the following beneficial effects.

Spectral reconstruction is performed on the satellite remote sensing data of the to-be-monitored area according to the UAV remote sensing data of the local area in the to-be-monitored area, so that the UAV remote sensing data of the local area are integrated into the reconstructed satellite spectral data of the satellite remote sensing data, and thus it is more accurate to determine the soil moisture content of the to-be-monitored area by using the reconstructed satellite spectral data of the satellite remote sensing data, and the timeliness is ensured, thereby realizing accurate and rapid soil moisture monitoring of the to-be-monitored area.

### BRIEF DESCRIPTION OF DRAWINGS

Accompanying drawings illustrated herein are provided to provide a further understanding of the present disclosure and constitute a part of the present disclosure. The illustrative embodiments of the present disclosure and the descriptions of the illustrative embodiments are used to explain the present disclosure and do not constitute an undue limitation of the present disclosure.

FIG. 1 illustrates a flow chart of a regional soil moisture monitoring method based on airborne spectrum reconstructed optical satellite remote sensing according to an embodiment of the present disclosure.

FIG. 2 illustrates a flow chart of step S102 according to an embodiment of the present disclosure.

FIG. 3 illustrates a schematic flow chart of another regional soil moisture monitoring method based on airborne spectrum reconstructed optical satellite remote sensing according to an embodiment of the present disclosure.

FIG. 4A illustrates a schematic matrix diagram of correlation analysis between original band reflectance and original spectral index and a soil moisture content according to an embodiment of the present disclosure.

FIG. 4B illustrates a schematic matrix diagram of correlation analysis between reconstructed band reflectance and reconstructed spectral index and a soil moisture content according to an embodiment of the present disclosure.

FIG. 5A illustrates a comparison diagram of scatter accuracy of a soil moisture inversion model under original satellite spectral data according to an embodiment of the present disclosure.

FIG. 5B illustrates a comparison diagram of scatter accuracy of a soil moisture inversion model under reconstructed satellite spectral data according to an embodiment of the present disclosure.

FIG. 6 illustrates a schematic view of a regional soil moisture monitoring device based on airborne spectrum reconstructed optical satellite remote sensing according to an embodiment of the present disclosure.

FIG. 7 illustrates a schematic view of computer device for implementing a regional soil moisture monitoring method based on airborne spectrum reconstructed optical satellite remote sensing according to an embodiment of the present disclosure.

### DETAILED DESCRIPTION OF EMBODIMENTS

In order to make objectives, technical solutions and advantages of the present disclosure clearly, the technical solutions of the present disclosure will be described clearly and completely with specific embodiments of the present disclosure and accompanying drawings. Apparently, the described embodiments are only parts of embodiments of the present disclosure, not the whole embodiment. Based on the embodiments in the present disclosure, all other embodiments obtained by ordinary technicians in the field without creative labor belong to the scope of protection of the present disclosure.

Multi-scale fusion of satellite remote sensing data and UAV remote sensing data can greatly improve the accuracy of soil moisture monitoring by satellite remote sensing. However, for a large-scale irrigation area, it is time-consuming and laborious to collect remote sensing data of UAV covering the whole large-scale irrigation area, and the timeliness of soil moisture monitoring products cannot be guaranteed.

Therefore, the disclosure provides a regional soil moisture monitoring method based on airborne spectrum reconstructed optical satellite remote sensing, which integrates the UAV remote sensing data of the local area into reconstructed satellite spectral data of the satellite remote sensing data to reconstruct the satellite remote sensing band reflectance and the satellite remote sensing spectral index, so as to improve the accuracy of satellite remote sensing monitoring of soil moisture and realize higher spatial-temporal resolution and higher precision monitoring of soil moisture in large-scale irrigation areas.

The technical solutions provided by various embodiment of the present disclosure will be described in detail below with reference to the accompanying drawings.

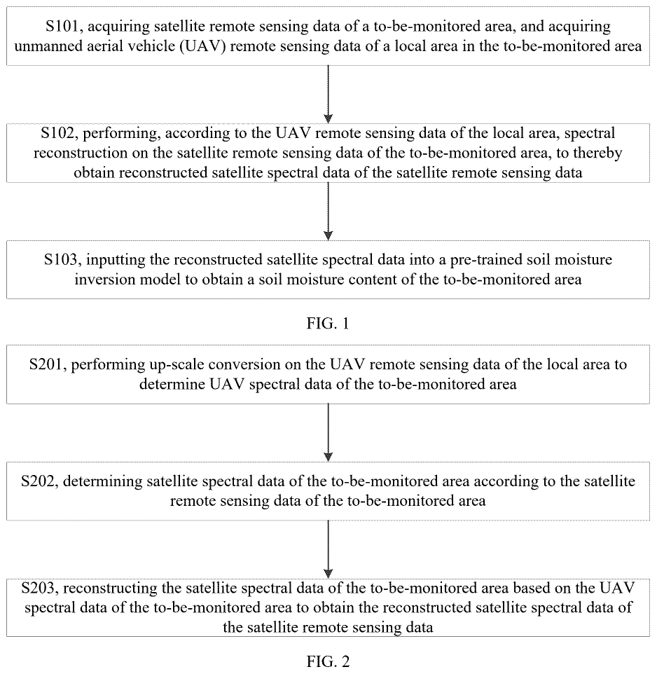

FIG. 1 illustrates a flow chart of a regional soil moisture monitoring method based on airborne spectrum reconstructed optical satellite remote sensing according to an embodiment of the present disclosure. The regional soil moisture monitoring method based on airborne spectrum reconstructed optical satellite remote sensing includes steps S101 through S103.

In step S101, satellite remote sensing data of a to-be-monitored area are acquired, and unmanned aerial vehicle (UAV) remote sensing data of a local area in the to-be-monitored area are acquired.

For example, the to-be-monitored area is an area whose soil moisture is to be monitored, and the local area is pre-defined area whose soil moisture is representative within the to-be-monitored area.

Remote sensing data of the to-be-monitored area can be collected directly by a satellite, and remote sensing data of the local area in the to-be-monitored area can be collected by an UAV. After the satellite acquires the remote sensing data of the to-be-monitored area, the satellite sends the remote sensing data of the to-be-monitored to a server to thereby obtain the satellite remote sensing data of the to-be-monitored area. After the UAV acquires the remote sensing data of the local area in the to-be-monitored area, the UAV sends the remote sensing data of the local area in the to-be-monitored area to the server to obtain the UAV remote sensing data of the local area in the to-be-monitored area.

The server mentioned in the present disclosure can be a server set on a business platform, or a device such as a desktop computer and a notebook computer that can implement the technical solutions of the present disclosure. For the convenience of explanation, the following description only takes the server as an execution subject.

In step SS102, spectral reconstruction is performed on the satellite remote sensing data of the to-be-monitored area according to the UAV remote sensing data of the local area, to thereby obtain reconstructed satellite spectral data of the satellite remote sensing data.

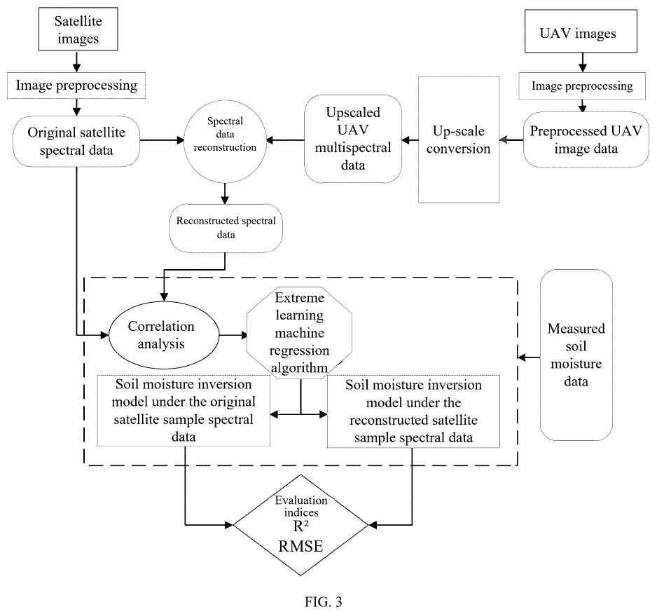

Specifically, as shown in FIG. 2, a process for performing, according to the UAV remote sensing data of the local area, the spectral reconstruction on the satellite remote sensing data of the to-be-monitored area, to thereby obtain the reconstructed satellite spectral data of the satellite remote sensing data includes steps S201 through S203.

In step 201, up-scale conversion is performed on the UAV remote sensing data of the local area to determine UAV spectral data of the to-be-monitored area.

Specifically, the UAV remote sensing data of the local area can be converted into the UAV spectral data of the to-be-monitored area by up-scaling conversion. An upscaling factor can be determined according to requirements, for example, from a resolution of 5 cm to a resolution of 10 m.

Optionally, the UAV remote sensing data of the local area can be input into a first learning model, and the first learning model is used for performing the up-scale conversion to obtain the UAV spectral data of the to-be-monitored area. Specifically, the UAV spectral data of the to-be-monitored area include spectral data of all areas in the to-be-monitored area. For example, in the first learning model, UAV spectral data of the local area can be determined according to the UAV remote sensing data of the local area, and then UAV spectral data of other areas of the to-be-monitored area can be obtained by learning the UAV spectral data of the local area, so as to obtain UAV spectral data of all areas in the to-be-monitored area.

Alternatively, the UAV remote sensing data of the local area can be input into a second learning model, and the second learning model is used for performing the up-scale conversion to obtain the UAV spectral data of the to-be-monitored area. Specifically, the UAV spectral data of the to-be-monitored area include spectral data of all areas in the to-be-monitored area. For example, in the second learning model, UAV remote sensing data of other areas of the to-be-monitored area can be obtained by learning the UAV remote sensing data of the local area, so as to obtain UAV remote sensing data of all areas in the to-be-monitored area; and then the UAV remote sensing data of all areas in the to-be-monitored area is analyzed to obtain the UAV spectral data of the to-be-monitored area.

In step S202, satellite spectral data of the to-be-monitored area are determined according to the satellite remote sensing data of the to-be-monitored area.

Specifically, the satellite remote sensing data of the to-be-monitored area may include the satellite spectral data of the to-be-monitored area.

In step 203, the satellite spectral data of the to-be-monitored area is reconstructed based on the UAV spectral data of the to-be-monitored area, to obtain the reconstructed satellite spectral data of the satellite remote sensing data.

In an embodiment, spectral data includes a band reflectance and a spectral index; and a process for reconstructing the satellite spectral data of the to-be-monitored area based on the UAV spectral data of the to-be-monitored area to obtain the reconstructed satellite spectral data of the satellite remote sensing data includes: reconstructing, based on the UAV spectral data of the to-be-monitored area, satellite band reflectance of the satellite spectral data of the to-be-monitored area to obtain a reconstructed satellite band reflectance of the reconstructed satellite spectral data of the satellite remote sensing data; and determining, based on the reconstructed satellite band reflectance of the reconstructed satellite spectral data of the satellite remote sensing data and a preset transformation function, a reconstructed satellite spectral index of the reconstructed satellite spectral data of the satellite remote sensing data.

In an embodiment, a formula for calculating the reconstructed satellite band reflectance of the reconstructed satellite spectral data of the satellite remote sensing data is expressed by a formula (1) as follows:

$${B}_{r} = {B}_{o} \{ 1 + k \cdot [ FVC / ( {B}_{u} \cdot {B}_{o} ) ] \} \qquad ( 1 )$$

where Bᵣ represents the reconstructed satellite band reflectance of the reconstructed satellite spectral data of the satellite remote sensing data; Bₒ represents a satellite band reflectance of the satellite spectral data of the to-be-monitored area; FVC represents a fractional vegetation coverage corresponding to a spatial resolution scale of an optical satellite; k represents a regulation factor, k∈R; and Bᵤ represents an UAV band reflectance or an UAV spectral index.

In an embodiment, the reconstructed satellite spectral index is a function of the reconstructed satellite band reflectance, and the preset transformation function is expressed by a formula (2) as follows:

$${S}_{rec} = f ( {B}_{r} ) \qquad ( 2 )$$

where S_{rec} represents the reconstructed satellite spectral index, f(.) represents a function from Bᵣ to S_{rec}, and the reconstructed satellite spectral index includes a vegetation index, a salinity index, a brightness index.

For example, as shown in Table 1, Table 1 shows the preset transformation function between the reconstructed satellite spectral index and the reconstructed satellite band reflectance.

TABLE 1

| | Spectral | Transformation |
|---|---|---|
| Type | index | function |
| Salinity index | SI | $\sqrt{B \cdot R}$ |
| | SI1 | $\sqrt{G \cdot R}$ |
| | SI2 | $\sqrt{{G}^{2} + {R}^{2} + {NIR}^{2}}$ |
| | SI3 | $\sqrt{{G}^{2} + {R}^{2}}$ |
| Vegetation index | NGBDI | $\frac{( G - B )}{( G + B )}$ |
| | DVI | NIR − R |
| | PDI | $\frac{( R + NIR )}{\sqrt{2}}$ |
| brightness index | BI | $\sqrt{{R}^{2} + {NIR}^{2}}$ |

where B, R, G and NIR represent wave band reflectances corresponding to Blue, Red, Green and near infrared (NIR), respectively.

In step S103, the reconstructed satellite spectral data are inputted into a pre-trained soil moisture inversion model to obtain a soil moisture content of the to-be-monitored area.

Specifically, the reconstructed satellite band reflectance and the reconstructed satellite spectral index are inputted into the pre-trained soil moisture inversion model to obtain the soil moisture content of the to-be-monitored area outputted from the pre-trained soil moisture inversion model.

In an embodiment, a construction process of the pre-trained soil moisture inversion model includes the following steps:
acquiring original satellite training sample data and UAV training sample data, and soil moisture contents corresponding to the original satellite training sample data and the UAV training sample data;
determining original satellite sample spectral data according to the original satellite training sample data;
performing, according to the UAV training sample data, spectral reconstruction on the original satellite sample spectral data, to thereby obtain reconstructed satellite sample spectral data; and
training an extreme learning machine model by using the reconstructed satellite sample spectral data, to obtain the pre-trained soil moisture inversion model.

Specifically, UAV multi-spectral remote sensing data, which are local and representative (different vegetation coverage conditions), optical satellite remote sensing images and soil moisture content data can be obtained and preprocessed. For example, the measured soil moisture contents and the UAV multi-spectral remote sensing data are obtained through field experiments, and satellite remote sensing data are obtained by downloading satellite remote sensing data from an official website. A number of pixels for up-scale conversion on the UAV remote sensing data of the local area is not less than 1000, and a number of soil moisture samples on the ground is not less than 120. The UAV multi-spectral remote sensing data is taken as UAV training sample data, and the optical satellite remote sensing images are taken as satellite training sample data.

Firstly, the satellite training sample data are spectrally reconstructed according to the UAV training sample data to obtain reconstructed satellite sample spectral data. It should be noted that the way to obtain the reconstructed satellite sample spectral data is the same as the way to obtain the reconstructed satellite spectral data of the satellite remote sensing data in the above embodiment, and the details of this embodiment are not repeated herein. The reconstructed satellite sample spectral data includes a reconstructed satellite sample band reflectance and a reconstructed satellite sample spectral index.

A k value in the reconstructed satellite sample band reflectance, i.e., the k in the above formula (1) is determined by constructing a linear correlation function r(k). The linear correlation function r(k) is constructed by the following formulas (3)-(6). The formula (3) is used to calculate the linear correlation coefficient r(Bᵣ, Sₘ) between the reconstructed satellite sample band reflectance and a corresponding soil moisture content.

$$r ( {B}_{r} , {S}_{m} ) = \frac{n {\sum }_{i = 1}^{n} ( {B}_{{r}_{i}} ) \cdot {s}_{{m}_{i}} - {\sum }_{i = 1}^{n} {B}_{{r}_{i}} \cdot {\sum }_{i = 1}^{n} {s}_{{m}_{i}}}{{[ n {\sum }_{i = 1}^{n} {( {B}_{{r}_{i}} )}^{2} - {( {\sum }_{i = 1}^{n} {B}_{{r}_{i}} )}^{2} ]}^{1 / 2} \cdot {[ n {\sum }_{i = 1}^{n} {s}_{{m}_{i}}^{2} - {( {\sum }_{i = 1}^{n} {s}_{{m}_{i}} )}^{2} ]}^{1 / 2}} \qquad ( 3 )$$

where Sₘ represents the soil moisture content, n represent a number of soil moisture samples, Bᵣ_{ᵢ} represents a reconstructed satellite band reflectance of an i-th sample of the n soil moisture samples, Sₘ_{ᵢ} represents a soil moisture content of the i-th sample of the n soil moisture samples.

$${M}_{0} = n {\sum }_{i = 1}^{n} {S}_{{m}_{i}}^{2} - {( {\sum }_{i = 1}^{n} {S}_{{m}_{i}} )}^{2} , ( {M}_{0} > 0 ) \qquad ( 4 )$$

$${M}_{1} = n {\sum }_{i = 1}^{n} {B}_{{o}_{i}} {S}_{{m}_{i}} - {\sum }_{i = 1}^{n} {B}_{{o}_{i}} \cdot {\sum }_{i = 1}^{n} {S}_{{m}_{i}} , ( {M}_{1} ∈ ℝ )$$

$${M}_{2} = n {\sum }_{i = 1}^{n} \frac{{FVC}_{i}}{{B}_{{u}_{i}}} {S}_{{m}_{i}} - {\sum }_{i = 1}^{n} \frac{{FVC}_{i}}{{B}_{{u}_{i}}} \cdot {\sum }_{i = 1}^{n} {S}_{{m}_{i}} , ( {M}_{2} ∈ ℝ )$$

$${M}_{3} = n {\sum }_{i = 1}^{n} {( \frac{{FVC}_{i}}{{B}_{{u}_{i}}} )}^{2} - {( {\sum }_{i = 1}^{n} \frac{{FVC}_{i}}{{B}_{{u}_{i}}} )}^{2} , ( {M}_{3} > 0 )$$

$${M}_{4} = n {\sum }_{i = 1}^{n} {B}_{{o}_{i}} \frac{{FVC}_{i}}{{B}_{{u}_{i}}} - {\sum }_{i = 1}^{n} {B}_{{o}_{i}} \cdot {\sum }_{i = 1}^{n} \frac{{FVC}_{i}}{{B}_{{u}_{i}}} , ( {M}_{4} ∈ ℝ )$$

$${M}_{5} = n {\sum }_{i = 1}^{n} {B}_{{o}_{i}}^{2} - {( {\sum }_{i = 1}^{n} {B}_{{o}_{i}} )}^{2} , ( {M}_{5} > 0 )$$

where Bₒ_{ᵢ} represents a satellite band reflectance of an i-th sample of the n soil moisture samples, Bᵤ_{ᵢ} represents an UAV band reflectance or an UAV spectral index of the i-th sample of the n soil moisture samples, and FVC; represents a fractional vegetation coverage of the i-th sample of the n soil moisture samples; and M₀, M₁, M₂, M₃, M₄, and M₅ are intermediate parameters and are used to simplify the formulas.

Based on the formula (4), the formula (3) is converted into the formula (5) expressed as follows:

$$r ( {B}_{r} , {S}_{m} ) = \frac{{M}_{2} \cdot k + {M}_{1}}{{( {M}_{0} {M}_{3} \cdot {k}^{2} + 2 {M}_{0} {M}_{4} \cdot k + {M}_{0} {M}_{5} )}^{1 / 2}} . \qquad ( 5 )$$

With a given number of soil moisture samples n, M₀, M₁, M₂, M₃, M₄, and M₅ are determined and are constants. Then the formula (5) can be expressed as the formula (6) expressed as follows:

$$r ( k ) = \frac{{M}_{2} \cdot k + {M}_{1}}{{( {M}_{0} {M}_{3} \cdot {k}^{2} + 2 {M}_{0} {M}_{4} \cdot k + {M}_{0} {M}_{5} )}^{1 / 2}} \qquad ( 6 )$$

where r(k) represents the linear correlation coefficient between the reconstructed satellite sample band reflectance and the corresponding soil moisture content, and r(k)∈[−1, 1].

Two sides of the formula (6) are differentiated with respect to k, and a formula (7) can be obtained, which is expressed as follows:

$$\frac{dr}{dk} = \frac{( {M}_{2} {M}_{4} - {M}_{1} {M}_{3} ) k + {M}_{2} {M}_{5} - {M}_{1} {M}_{4}}{{{M}_{0}^{1 / 2} ( {M}_{3} {k}^{2} + 2 {M}_{4} k + {M}_{5} )}^{3 / 2}} \qquad ( 7 )$$

The formula (7) has an extreme value (maximum value) of the linear correlation coefficient r(k), so that dr/dk=0 in the formula (7), and thus a formula (8) is obtained, which is expressed as follows:

$${k}_{m} = \frac{{M}_{1} {M}_{4} - {M}_{2} {M}_{5}}{{M}_{2} {M}_{4} - {M}_{1} {M}_{3}} , ( {M}_{2} {M}_{4} \neq {M}_{1} {M}_{3} ) . \qquad ( 8 )$$

With respect to the linear correlation coefficient r(k), there are four special cases (i), (ii), (iii) and (iv).

(i) When k=(M₁M₄-M₂M₅)/(M₂M₄-M₁M₃) in the formula (6), a formula (9) is determined, which is expressed as follows:

$${r}_{e} = \frac{{M}_{2} \frac{{M}_{1} {M}_{4} - {M}_{2} {M}_{5}}{{M}_{2} {M}_{4} - {M}_{1} {M}_{3}} + {M}_{1}}{{{M}_{0} [ {{M}_{3} ( \frac{{M}_{1} {M}_{4} - {M}_{2} {M}_{5}}{{M}_{2} {M}_{4} - {M}_{1} {M}_{3}} )}^{2} + 2 {M}_{4} \frac{{M}_{1} {M}_{4} - {M}_{2} {M}_{5}}{{M}_{2} {M}_{4} - {M}_{1} {M}_{3}} + {M}_{5} ]}^{1 / 2}} \qquad ( 9 )$$

where (kₑ, rₑ) represents a unique extreme point of the function r(k) in areal number range, and rₑ represents a unique extreme value of the function r(k).

(ii) When k=0 in the formula (6), a formula (10) is obtained, which is expressed as follows:

$${r}_{o} = \frac{{M}_{1}}{\sqrt{{M}_{0} \cdot {M}_{5}}} \qquad ( 10 )$$

where rₒ represents a linear correlation coefficient between Bₒ and Sₘ, that is, the correlation coefficient between original satellite band reflectance and soil moisture.
(iii) When k=-M₁/M₂ in the formula (6), r(k)=0, i.e., there is no linear correlation between the reconstructed satellite band reflectance and soil moisture.

(iv) When k→∞ in the formula (6), a formula (11) is determined, which is expressed as follows:

$${r}_{∞} = {lim}_{k \to \pm ∞} r ( k ) = \frac{{M}_{2}}{{( {M}_{0} \cdot {M}_{3} )}^{1 / 2}} \qquad ( 11 )$$

where r, represents a linear correlation coefficient between B, and s, at positive and negative infinity.

The reconstructed satellite sample spectral data includes a reconstructed satellite sample band reflectance and a reconstructed satellite sample spectral index. According to the km calculated in the formula (8), an original satellite band reflectance Bₒ corresponding to the satellite training sample data, the vegetation coverage FVC, and the UAV multispectral band reflectance or UAV multispectral spectral index Bᵤ corresponding to upscaled UAV training sample data, the reconstructed satellite sample band reflectance can be calculated based on the formula (1), and the reconstructed satellite sample spectral index can be calculated based on the reconstructed satellite sample band reflectance.

After the reconstructed satellite sample spectral data are obtained, an extreme learning machine model is trained by the reconstructed satellite sample spectral data, to thereby obtain a soil moisture inversion model under the reconstructed satellite sample spectral data. Specifically, machine learning algorithms for an extreme learning machine are used to construct a soil moisture inversion regression model of the reconstructed satellite sample spectral data, to obtain the soil moisture inversion model.

In an exemplary embodiment, this embodiment includes: before training the extreme learning machine model, a correlation analysis is performed on the original satellite sample spectral data and the reconstructed satellite sample spectral data with the soil moisture contents, respectively. For example, Pearson correlation is used in the correlation analysis.

Moreover, the method includes: training the extreme learning machine model by using the original satellite sample spectral data, to obtain a soil moisture inversion model under the original satellite sample spectral data; training the extreme learning machine model by using the reconstructed satellite sample spectral data, to obtain a soil moisture inversion model under the reconstructed satellite sample spectral data; and performing performance testing on the soil moisture inversion model under the original satellite sample spectral data and the soil moisture inversion model under the reconstructed satellite sample spectral data, to obtain an original performance test result of the soil moisture inversion model under the original satellite sample spectral data and a performance test result of the soil moisture inversion model under the reconstructed satellite sample spectral data.

In an embodiment, each of the soil moisture inversion model under the original satellite sample spectral data and the soil moisture inversion model under the reconstructed satellite sample spectral data is an inversion regression model.

Optionally, a precision of the soil moisture inversion model under the original satellite sample spectral data and a precision of the soil moisture inversion model under the reconstructed satellite sample spectral data are evaluated by a determination coefficient (R²) and a root mean square error (RMSE), and increase or decrease ranges of R² and RMSE are used to evaluate an improvement effect of the soil moisture inversion model under the reconstructed satellite sample spectral data compared with the soil moisture inversion model under the original satellite sample spectral data.

In an exemplary embodiment, as shown in FIG. 3, the present disclosure also provides a regional soil moisture monitoring method based on airborne spectrum reconstructed optical satellite remote sensing. Specifically, the method includes: acquiring satellite images of a sample area collected by a satellite; preprocessing the satellite images to obtain original satellite spectral data; acquiring UAV images of a sample area collected by an UAV; preprocessing the UAV images to obtain preprocessed UAV image data; and performing up-scale conversion on the UAV image data to obtain upscaled UAV multispectral data.

Then, the original satellite spectral data are then spectrally reconstructed using the upscaled UAV multispectral data to obtain reconstructed satellite spectral data. Correlation analysis is performed between the original satellite spectral data and measured soil moisture contents, and correlation analysis is further performed between the reconstructed satellite spectral data and the measured soil moisture contents. Subsequently, the measured soil moisture contents are taken as dependent variables with the original satellite spectral data and the reconstructed satellite spectral data as independent variables, a soil moisture inversion model under the original satellite spectral data and a soil moisture inversion model under the reconstructed satellite spectral data are trained, and then the soil moisture inversion model under the original satellite spectral data and the soil moisture inversion model under the reconstructed satellite spectral data are evaluated by evaluation indices (R² and RMSE), and a corresponding evaluation result indicates that the performance of the soil moisture inversion model under the reconstructed satellite spectral data is better compared with the soil moisture inversion model under the original satellite spectral data.

The regional soil moisture monitoring method based on airborne spectrum reconstructed optical satellite remote sensing provided by the present disclosure will be further explained through a specific implementation.

Specifically, local UAV multispectral remote sensing data (four fields with different vegetation coverage are selected) from a jiefangzha shahao canal irrigation district in a hetao irrigation area of a region A, measured soil moisture data of four typical fields collected simultaneously, and downloaded cloud-free Landsat 8 satellite multispectral remote sensing data corresponding to a study area are used to construct an inversion model for soil moisture content in irrigation area based on original satellite spectral data and an inversion model for soil moisture content in irrigation area based on constructed satellite spectral data based on UAV spectrum, and their inversion accuracies and improvement effects are compared through evaluation indices. The method includes a first step, a second step, a third step, a fourth step, a fifth step, and a sixth step.

In the first step, soil sample collection and an UAV flight test were carried out for the four selected typical fields on Aug. 12-15, 2019. Visible light bands of UAV multispectral data, namely a blue band, a green band and a red band, are used for up-scaling conversion. Soil samples with a depth of 0-10 cm were collected by a five-point sampling method. A mass moisture content Sₘ of soil is calculated by a drying method (constant temperature treatment at a temperature of 105° C. for 24 h), and a calculation formula for the mass moisture content Sₘ of soil is as follows:

$${S}_{m} = \frac{{m}_{1} - {m}_{2}}{{m}_{2} - {m}_{3}} \times 100 \% \qquad ( 12 )$$

where m₁ represents a weight of wet soil and an empty aluminum box, m₂ represents a weight of dry soil and the empty aluminum box, and m₃ represents a weight of the empty aluminum box.

Landsat 8 multispectral satellite remote sensing data were downloaded from united states geological survey (USGS) Earth Explorer, and the acquired Landsat 8 satellite images were geometrically corrected Collection2 Level-1 products. An imaging time of the image was Aug. 15, 2019, which was close to a sampling time in the field. The Landsat 8 satellite image data were further processed by Environment for Visualizing Images (ENVI) software, and the specific processes include radiometric calibration, atmospheric correction, and cropping, and then reflectance data of each band at sampling points are extracted. In the present disclosure, Blue, Green, Red, near infrared (NIR), short-wave infrared-1 (SWIR1) and short-wave infrared-2 (SWIR2) of Landsat 8 optical satellite data were selected for reconstruction.

In the second step, FVC is obtained by extracting proportions of vegetation elements in a high-resolution UAV multispectral image at a given scale (30 m), and a calculation formula therefor is as follows.

$$FVC = \frac{{N}_{v}}{{N}_{v} + {N}_{no \_ v}} \qquad ( 13 )$$

where Nᵥ and Nₙₒ₋ᵥ represent a number of vegetation pixels and a number of non-vegetation pixels corresponding to a spatial resolution scale of Landsat 8 satellite, respectively.

In the present disclosure, a salinity index S3, i.e. S3ᵤ, calculated based on upscaled UAV remote sensing data is determined as Bᵤ, and a calculation formula for the S3u is as follows:

$$S {3}_{u} = {Green}_{u} \times {Red}_{u} / {Blue}_{u} \qquad ( 14 )$$

where Blueᵤ, Greenᵤ, and Redᵤ are blue, green and red band reflectances of UAV remote sensing data, respectively, and the UAV remote sensing data have been upscaled to a 30-meter spatial resolution, and an up-scale conversion method herein is a pixel aggregation method.

A formula for k value at the extreme point of the linear function r(k) is used to calculate a kₘ value of each reconstructed band reflectance, namely, k_{m-Blue}, k_{m-Green}, k_{m-Red}, k_{m-NIR}, k_{m-SWIR1}, and k_{m-SWIR2}, and finally each reconstructed band reflectance is obtained by the formulas (15)-(20).

$${Blue}_{re} = Blue \{ 1 + {k}_{m - Blue} \cdot [ \frac{FVC}{S {3}_{u} \cdot Blue} ] \} \qquad ( 15 )$$

$${Green}_{re} = Green \{ 1 + {k}_{m - Green} \cdot [ \frac{FVC}{S {3}_{u} \cdot Green} ] \} \qquad ( 16 )$$

$${Red}_{re} = Red \{ 1 + {k}_{m - Red} \cdot [ \frac{FVC}{S {3}_{u} \cdot Red} ] \} \qquad ( 17 )$$

$${NIR}_{re} = NIR \{ 1 + {k}_{m - NIR} \cdot [ \frac{FVC}{S {3}_{u} \cdot NIR} ] \} \qquad ( 18 )$$

$$SWIR {1}_{re} = SWIR 1 \{ 1 + {k}_{m - SWIR 1} \cdot [ \frac{FVC}{S {3}_{u} \cdot SWIR 1} ] \} \qquad ( 19 )$$

$$SWIR {2}_{re} = SWIR 2 \{ 1 + {k}_{m - SWIR 2} \cdot [ \frac{FVC}{S {3}_{u} \cdot SWIR 2} ] \} \qquad ( 20 )$$

In the third step, eight original spectral indices (including four salinity indices, three vegetation indices, and one brightness index) are constructed based on the band reflectance of the original Landsat 8 optical satellite data; and eight reconstructed spectral indices (including four reconstructed salinity indices, three reconstructed vegetation indices, and one reconstructed brightness index) are constructed based on the band reflectance of the reconstructed Landsat 8 optical satellite data. The specific calculation formulas are shown in Table 2.

TABLE 2

| | Original | Calculation | Constructed | Calculation |
|---|---|---|---|---|
| Type | index | formula | index | formula |
| Salinity | SI | $\sqrt{B \cdot R}$ | SIᵣₑ | $\sqrt{{B}_{re} \cdot {R}_{re}}$ |
| index | SI1 | $\sqrt{G \cdot R}$ | SI1ᵣₑ | $\sqrt{{G}_{re} \cdot {R}_{re}}$ |
| | SI2 | $\sqrt{{G}^{2} + {R}^{2} + {NIR}^{2}}$ | SI2ᵣₑ | $\sqrt{{G}_{re}^{2} + {R}_{re}^{2} + {NIR}_{re}^{2}}$ |
| | SI3 | $\sqrt{{G}^{2} + {R}^{2}}$ | SI3ᵣₑ | $\sqrt{{G}_{re}^{2} + {R}_{re}^{2}}$ |
| Vegetation index | NGBDI | $\frac{( G - B )}{( G + B )}$ | NGBDIᵣₑ | $\frac{( {G}_{re} - {B}_{re} )}{( {G}_{re} + {B}_{re} )}$ |
| | DVI | NIR − R | DVIᵣₑ | NIRᵣₑ − Rᵣₑ |
| | PDI | $\frac{( R + NIR )}{\sqrt{2}}$ | PDIᵣₑ | $\frac{( {R}_{re} + {NIR}_{re} )}{\sqrt{2}}$ |
| Brightness index | | $\sqrt{{R}^{2} + {NIR}^{2}}$ | BIᵣₑ | $\sqrt{{R}_{re}^{2} + {NIR}_{re}^{2}}$ |

Specifically, B, G, R and NIR respectively represent an original blue band reflectance, an original green band reflectance, an original red band reflectance and an original near infrared band reflectance, that is, B=Blue, G-Green and R=Red; Bᵣₑ, Gᵣₑ, Rᵣₑ, and NIRᵣₑ respectively represent a reconstructed blue band reflectance, a reconstructed green band reflectance, a reconstructed red band reflectance, and a reconstructed near infrared band reflectance, that is, Bᵣₑ=Blueᵣₑ, Gᵣₑ-Greenᵣₑ, and Rᵣₑ=Redᵣₑ.

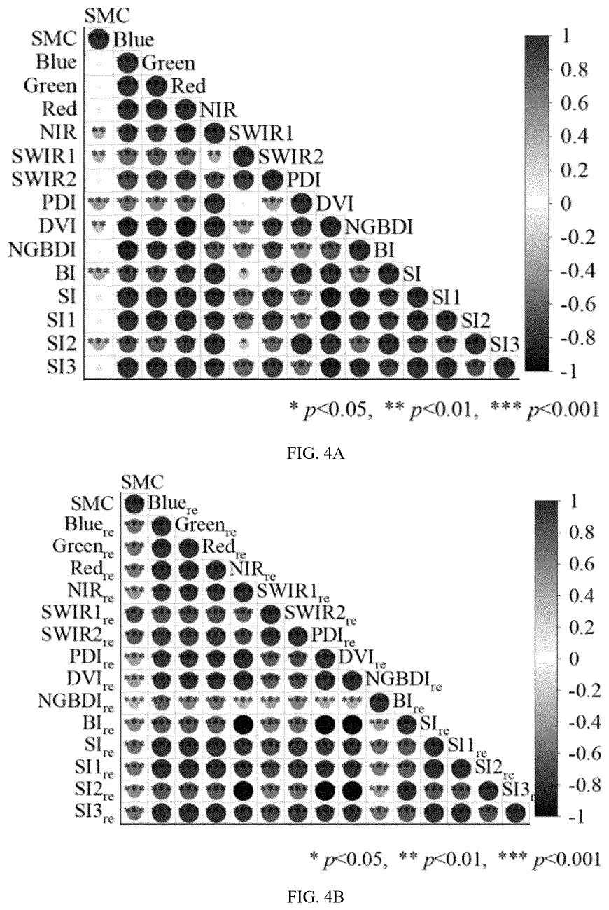

In the fourth step, correlation analysis is performed on the measured soil moisture content and the original satellite band reflectances and the original satellite spectral indices, and correlation analysis is performed on the measured soil moisture content and the reconstructed satellite band reflectances and the reconstructed satellite spectral indices, and the results are shown by a Pearson correlation matrix diagram, as shown in FIG. 4A and FIG. 4B, where the larger the circle, the stronger the correlation. As can be seen from FIG. 4 and FIG. 4B, compared with the original satellite band reflectances and the original satellite spectral indices, the correlation between the reconstructed satellite band reflectances and the reconstructed satellite spectral indices and the soil moisture content is significantly enhanced.

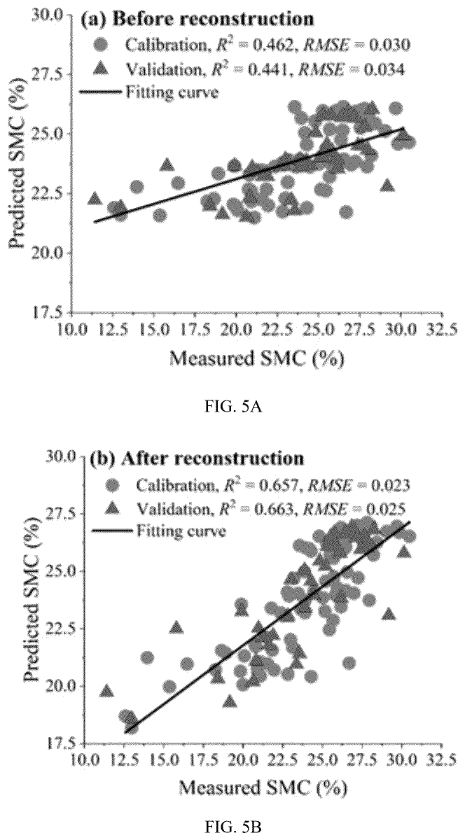

In the fifth step, an extreme learning machine is an artificial neural network model training algorithm, which usually includes an input layer, a hidden layer and an output layer. In the process of model training, connection weights of the input layer and the hidden layer are random and do not need to be adjusted, and a global optimal solution can be generated only by setting the number of nodes of the hidden layer of the network. In the present disclosure, the number of nodes of the hidden layer is uniformly set to 20, and the extreme learning machine model is built by a MATrix LABoratory (MATLAB) software. A sample number ratio of a modeling set and a verification set is set to 2:1, that is, a sample number of modeling set is 79, and a sample number of verification set is 40. Taking all original band reflectances and original spectral indices as independent variables and the soil moisture as a dependent variable, a soil moisture inversion model under the original satellite data is constructed by using an extreme learning machine regression algorithm. Taking all reconstructed band reflectances and reconstructed spectral indices as independent variables and the soil moisture as a dependent variable, a soil moisture inversion model under the reconstructed satellite data is constructed by an extreme learning machine regression algorithm, and the results are shown in FIG. 5A and FIG. 5B.

In the sixth step, a determination coefficient can reflect a fitting effect of the model. The closer R² is to 1, the smaller RMSE is, indicating that the better the prediction effect of the model is, and the smaller an error between the predicted value and the measured value is. R² and RMSE between the measured and predicted values of soil moisture content are calculated, as shown in Table 3.

$${R}^{2} = 1 - \frac{{\sum }_{i = 1}^{n} {( {y}_{i} - {\hat{y}}_{i} )}^{2}}{{\sum }_{i = 1}^{n} {( {y}_{i} - \overline{y} )}^{2}} \qquad ( 21 )$$

$$RMSE = \sqrt{\frac{1}{n} {\sum }_{i = 1}^{n} {( {\hat{y}}_{i} - {y}_{i} )}^{2}} \qquad ( 22 )$$

where yᵢ represents the measured value, ŷᵢ represents the predicted value, \tilde{y}ᵢ represents an average value of the measured value, and n represents a number of samples.

TABLE 3
Effect table before and after reconstruction of satellite spectral data

| Accuracy index | Modeling data | Verification data |
|---|---|---|
| R² | +42.21% | +50.34% |
| RMSE | −23.33% | −26.47% |

It should be noted that the results in Table 3 show the increase and decrease of R² and RMSE of the extreme learning machine model constructed with the reconstructed satellite spectral data compared with the extreme learning machine model constructed with the original satellite spectral data. Specifically, + indicates rising, and − indicates decreasing.

Combined with FIGS. 5A and 5B and Table 3, it can be seen that R² of a validation set of a soil moisture inversion model constructed by using the original satellite data is 0.441, and RMSE of a validation set of the soil moisture inversion model constructed by using the original satellite data is 0.034 cm³/cm³. In contrast, R² of a validation set of a soil moisture inversion model under the reconstructed satellite data constructed by the extreme learning machine regression algorithm is 0.663, and RMSE of a validation set of a soil moisture inversion model under the reconstructed satellite data constructed by the extreme learning machine regression algorithm is 0.025 cm³/cm³. R² is increased by 0.222 (about 50.34%) and the RMSE is decreased by 0.009 cm³/cm³ (about 26.47%). It can be seen that the integration of UAV multi-spectral data of the local area can effectively enhance the accuracy of soil moisture inversion models based solely on satellite optical remote sensing, improve the accuracy of soil moisture monitoring by satellite remote sensing, and realize higher spatial-temporal resolution and higher precision monitoring of soil moisture in large-scale irrigation areas. The present disclosure can provide theoretical basis and technical support for improving the monitoring accuracy of regional soil moisture content based on satellite remote sensing spectral data.

When applying the regional soil moisture monitoring method based on airborne spectrum reconstructed optical satellite remote sensing provided by the present disclosure, it may not be executed according to the sequence of each step shown in FIG. 1, and the specific execution sequence of each step may be determined as required, and the present disclosure does not limit this.

Further, the regional soil moisture monitoring method further includes the following steps:
converting the soil moisture content of the to-be-monitored area into the relative moisture content (RWC) to eliminate the difference of soil types by using the following formula:

RWC=(soil moisture content of the to-be-monitored area−withering coefficient)/(field capacity−withering coefficient)×100%
where the wilting coefficient and the field capacity can be obtained from regional soil type databases, such as American SSURGO soil database or China soil records;
determining a drought level of the to-be-monitored area based on the following table:

| Drought level | RWC | Representative Meaning |
|---|---|---|
| No drought | 60%~100% | Sufficient soil moisture for |
| | | optimal crop growth |
| Mild drought | 40%~60% | Slight water stress impairing |
| | | crop development. |
| Moderate drought | 20%~40% | Visible moisture stress |
| | | indicated by leaf wilting |
| Severe drought | <20% | Acute water stress resulting in |
| | | significant yield loss |

re-classifying normalized relative moisture content grid data according to thresholds in the above table, and obtaining a spatial distribution grid map of a drought level of the to-be-monitored area, which can directly display a location and scope of drought.

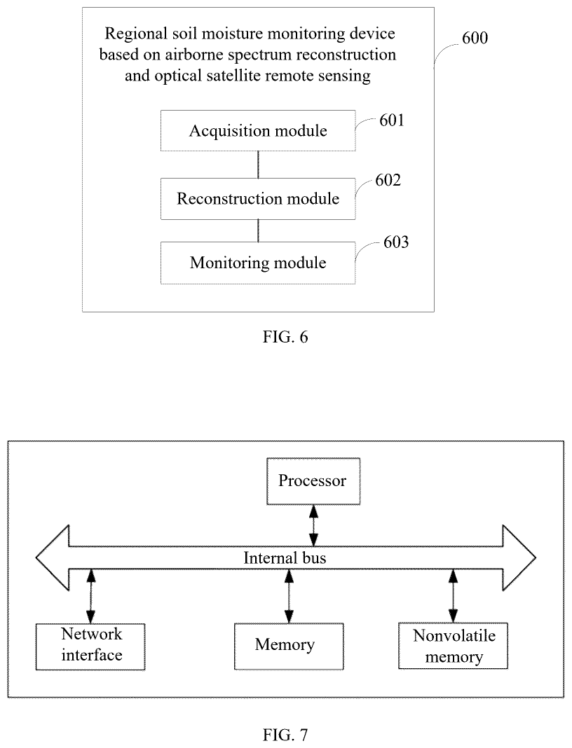

The above is the regional soil moisture monitoring method based on airborne spectrum reconstructed optical satellite remote sensing provided by one or more embodiments of the present disclosure. Based on the same idea, the present disclosure also provides a regional soil moisture monitoring device based on airborne spectrum reconstructed optical satellite remote sensing, as shown in FIG. 6.

FIG. 6 illustrates a schematic view of a regional soil moisture monitoring device based on airborne spectrum reconstructed optical satellite remote sensing according to an embodiment of the present disclosure. The device 600 includes: an acquisition module 601, a reconstruction module 602, and a monitoring module 603.

The acquisition module 601 is configured to: acquire satellite remote sensing data of a to-be-monitored area, and acquire unmanned aerial vehicle (UAV) remote sensing data of a local area in the to-be-monitored area.

The reconstruction module 602 is configured to: perform, according to the UAV remote sensing data of the local area, spectral reconstruction on the satellite remote sensing data of the to-be-monitored area, to thereby obtain reconstructed satellite spectral data of the satellite remote sensing data.

The monitoring module 603 is configured to: input the reconstructed satellite spectral data into a pre-trained soil moisture inversion model to obtain a soil moisture content of the to-be-monitored area.

For the specific definition of regional soil moisture monitoring device based on airborne spectrum reconstructed optical satellite remote sensing, please refer to the above definition of the regional soil moisture monitoring method based on airborne spectrum reconstructed optical satellite remote sensing, and will not be repeated here. Each module in the above-mentioned regional soil moisture monitoring device based on airborne spectrum reconstructed optical satellite remote sensing can be realized in whole or in part by software, hardware and their combinations. The above modules can be embedded in or independent of a processor in computer device in the form of hardware, and can also be stored in a memory in the computer device in the form of software, so that the processor can call and execute the operations corresponding to the above modules.

The disclosure also provides a computer-readable storage medium, which stores a computer program, and the computer program can be used for executing the regional soil moisture monitoring method based on airborne spectrum reconstructed optical satellite remote sensing in FIG. 1.

The disclosure also provides a schematic structural diagram of the computer device shown in FIG. 7. As shown in FIG. 7, in the hardware level, the computer device includes a processor, an internal bus, a network interface, a memory and a nonvolatile memory, and of course, it may also include hardware required by other services. The processor reads the corresponding computer program from the non-volatile memory into the memory and runs it, to realize the regional soil moisture monitoring method based on airborne spectrum reconstructed optical satellite remote sensing in FIG. 1.

Those skilled in the art can understand that all or part of the processes in the method for realizing the above-mentioned embodiments can be completed by instructing related hardware through a computer program, which can be stored in a nonvolatile computer-readable storage medium, and when executed, the computer program can include the processes of the above-mentioned embodiments. Any reference to memory, storage, database or other media used in various embodiments provided by the present disclosure may include at least one of nonvolatile and volatile memory. Non-volatile memory may include a read-only memory (ROM), a magnetic tape, a floppy disk, a flash memory or an optical memory, and the like. The volatile memory may include a random access memory (RAM) or an external cache. By way of illustration and not limitation, RAM can be in various forms, such as a static random access memory (SRAM) or a dynamic random access memory (DRAM).

The technical features of the above embodiments can be combined at will. In order to make the description concise, not all possible combinations of the technical features in the above embodiments are described. However, as long as there is no contradiction between the combinations of these technical features, they should be considered as the scope of protection of the present disclosure.

What is claimed is:

1. A regional soil moisture monitoring method based on airborne spectrum reconstructed optical satellite remote sensing, comprising the following steps:
acquiring satellite remote sensing data of a to-be-monitored area, and acquiring unmanned aerial vehicle (UAV) remote sensing data of a local area in the to-be-monitored area;
performing, according to the UAV remote sensing data of the local area, spectral reconstruction on the satellite remote sensing data of the to-be-monitored area, to obtain reconstructed satellite spectral data of the satellite remote sensing data, wherein the reconstructed satellite spectral data comprises a reconstructed satellite band reflectance, and a formula for calculating the reconstructed satellite band reflectance is expressed as follows:
$${B}_{r} = {B}_{o} \{ 1 + k \cdot [ FVC / ( {B}_{u} \cdot {B}_{o} ) ] \}$$
where B, represents the reconstructed satellite band reflectance; Bₒ represents a satellite band reflectance; FVC represents a fractional vegetation coverage corresponding to a spatial resolution scale of an optical satellite; k represents a regulation factor, k∈R; and Bᵤ represents an UAV band reflectance or an UAV spectral index; and
inputting the reconstructed satellite spectral data into a pre-trained soil moisture inversion model to obtain a soil moisture content of the to-be-monitored area.

2. The method as claimed in claim 1, wherein the performing, according to the UAV remote sensing data of the local area, spectral reconstruction on the satellite remote sensing data of the to-be-monitored area, to thereby obtain reconstructed satellite spectral data of the satellite remote sensing data comprises:
performing up-scale conversion on the UAV remote sensing data of the local area to determine UAV spectral data of the to-be-monitored area;
determining satellite spectral data of the to-be-monitored area according to the satellite remote sensing data of the to-be-monitored area; and
reconstructing the satellite spectral data of the to-be-monitored area based on the UAV spectral data of the to-be-monitored area to obtain the reconstructed satellite spectral data of the satellite remote sensing data.

3. The method as claimed in claim 2, wherein the reconstructed satellite spectral data further comprises a reconstructed satellite spectral index; and the reconstructing the satellite spectral data of the to-be-monitored area based on the UAV spectral data of the to-be-monitored area to obtain the reconstructed satellite spectral data of the satellite remote sensing data comprises:
reconstructing, based on the UAV spectral data of the to-be-monitored area, the satellite band reflectance to obtain the reconstructed satellite band reflectance; and
determining, based on the reconstructed satellite band reflectance and a preset transformation function, the reconstructed satellite spectral index.

4. The method as claimed in claim 1, wherein a construction process of the pre-trained soil moisture inversion model comprises the following steps:
acquiring original satellite training sample data and UAV training sample data, and soil moisture contents corresponding to the original satellite training sample data and the UAV training sample data;
determining original satellite sample spectral data according to the original satellite training sample data;
performing, according to the UAV training sample data, spectral reconstruction on the original satellite sample spectral data, to obtain reconstructed satellite sample spectral data; and
training an extreme learning machine model by using the reconstructed satellite sample spectral data, to obtain the pre-trained soil moisture inversion model.

5. The method as claimed in claim 4, further comprising:
before training the extreme learning machine model by using the reconstructed satellite sample spectral data, to obtain the pre-trained soil moisture inversion model, performing a correlation analysis on each of the original satellite sample spectral data and the reconstructed satellite sample spectral data with the soil moisture contents.

6. The method as claimed in claim 4, further comprising:
training the extreme learning machine model by using the original satellite sample spectral data, to obtain a soil moisture inversion model under the original satellite sample spectral data;
training the extreme learning machine model by using the reconstructed satellite sample spectral data, to obtain a soil moisture inversion model under the reconstructed satellite sample spectral data; and
performing performance testing on the soil moisture inversion model under the original satellite sample spectral data and the soil moisture inversion model under the reconstructed satellite sample spectral data, to obtain an original performance test result of the soil moisture inversion model under the original satellite sample spectral data and a performance test result of the soil moisture inversion model under the reconstructed satellite sample spectral data.

7. A regional soil moisture monitoring device based on airborne spectrum reconstructed optical satellite remote sensing, comprising:
an acquisition module, configured to: acquire satellite remote sensing data of a to-be-monitored area, and acquire unmanned aerial vehicle (UAV) remote sensing data of a local area in the to-be-monitored area;
a reconstruction module, configured to: perform, according to the UAV remote sensing data of the local area, spectral reconstruction on the satellite remote sensing data of the to-be-monitored area, to obtain reconstructed satellite spectral data of the satellite remote sensing data, wherein the reconstructed satellite spectral data comprises a reconstructed satellite band reflectance, and a formula for calculating the reconstructed satellite band reflectance is expressed as follows:
$${B}_{r} = {B}_{o} \{ 1 + k \cdot [ FVC / ( {B}_{u} \cdot {B}_{o} ) ] \}$$
where B, represents the reconstructed satellite band reflectance; Bₒ represents a satellite band reflectance; FVC represents a fractional vegetation coverage corresponding to a spatial resolution scale of an optical satellite; k represents a regulation factor, k∈R; and Bᵤ represents an UAV band reflectance or an UAV spectral index; and
a monitoring module, configured to: input the reconstructed satellite spectral data into a pre-trained soil moisture inversion model to obtain a soil moisture content of the to-be-monitored area.

8. A computer-readable storage medium, wherein a computer program is stored in the computer-readable storage medium, and when the computer program is executed by a processor, the regional soil moisture monitoring method based on airborne spectrum reconstructed optical satellite remote sensing according to claim 1 is implemented.

9. A computer device, comprising: a memory, a processor and a computer program stored in the memory and capable of being executed by the processor, and when the computer program is executed by the processor, the regional soil moisture monitoring method based on airborne spectrum reconstructed optical satellite remote sensing according to claim 1 is implemented.
